(19) United States
(12) Patent Application Publication
(10) Pub. No.: US 2026/0137898 A1
(43) Pub. Date: May 21, 2026

(54) METHOD AND SYSTEM FOR INDUCING SLEEP POSTURE TO PREVENT SNORING AND SLEEP APNEA DURING SLEEP

(71) Applicant: SB Solutions Inc., Ulsan (KR)

(72) Inventor: Seungup SEO, Ulsan (KR)

(73) Assignee: SB Solutions Inc., Ulsan (KR)

(21) Appl. No.: 19/405,159

(22) Filed: Dec. 1, 2025

Related U.S. Application Data
(63) Continuation of application No. PCT/KR2024/005418, filed on Apr. 22, 2024.

(30) Foreign Application Priority Data
May 31, 2023 (KR) 10-2023-0070085

(51) Int. Cl. A61M 21/02; A61B 5/00; A61B 5/11; A61M 21/00

(52) U.S. Cl. CPC A61M 21/02; A61B 5/1116; A61B 5/4818; A61B 5/6801; A61M 2021/0022

(57) ABSTRACT

A method and a system for inducing a sleep posture to prevent snoring and sleep apnea during sleep are disclosed. A system for inducing sleep posture according to one embodiment may comprise a sensor device attached to a body of a user, the sensor device being configured to collect first data comprising at least one of posture data of the user and respiration data of the user and transmit the first data to a user device, and to generate vibration for inducing a change in a sleep posture of the user in response to a control signal received from the user device; and the user device configured to collect second data comprising sound information related to sleep of the user, to aggregate and analyze the first data received from the sensor device and the second data to determine a snoring state or a sleep apnea state of the user, and to transmit, to the sensor device, the control signal for generation of vibration in response to a determination that the user is in the snoring state or the sleep apnea state.

### CROSS-REFERENCE TO RELATED APPLICATIONS

This U.S. non-provisional application is a continuation of PCT International Application No. PCT/KR 2024/005418, filed on Apr. 22, 2024, which claims priority under 35 U.S.C. 119 to Korean Patent Application No. 10-2023-0070085, filed with the Korean Intellectual Property Office on May 31, 2023. The disclosures of the foregoing applications are incorporated herein by reference in their entirety.

### BACKGROUND OF THE INVENTION

Snoring not only interferes with restful sleep of others but may also cause health problems for the individual who snores. In the Republic of Korea, the number of people who habitually snore is estimated to be ten million, meaning that approximately one out of every five people snores. Snoring occurs when an upper airway, which is a space for breathing and includes a nasal cavity, a pharynx, and a larynx, becomes narrowed or obstructed. In general, as a person ages, elasticity of muscles in the airway decreases and the muscles become lax, or structures around the airway increase due to obesity, thereby narrowing the airway. In addition, snoring may be caused when a root of a tongue is pushed toward the airway due to a small jaw structure or space, or when a tongue is congenitally large and obstructs the airway. When breathing occurs while the upper airway is narrowed due to various causes, vibration of a tongue, a throat, and a palate generates sound.

Snoring should not be neglected by merely regarding it as a loud sound that disturbs sleep. This is because severe snoring may lead to sleep apnea. Sleep apnea refers to a condition in which breathing stops for 10 seconds or longer during sleep 5 times or more per hour, or 30 times or more over 7 hours. Even when sufficient sleep time is obtained, unexplained chronic fatigue symptoms may accompany the condition. In addition, when hypertension persists in a person who snores, or when diabetes is not properly treated, sleep apnea may be suspected. Along with this, symptoms such as extreme fatigue that cannot be endured without daytime napping, severe morning headaches, unexplained dizziness, decreased libido, and erectile dysfunction may occur.

Sleep apnea makes oxygen supply within a body difficult and causes various problems. When hypoxemia occurs due to improper breathing during sleep for various reasons, myocardial infarction, hypertension, and stroke may result. In particular, when sleep apnea occurs, a body exerts effort to exhale obstructed breath in order to ensure smooth oxygen supply, and during this process, risks of stroke and hypertension are known to further increase. In addition, when sleep is repeatedly interrupted, an autonomic nervous system is stimulated, thereby increasing a risk of heart disease. According to research results, people who frequently experience snoring and sleep apnea are also vulnerable to chronic bronchitis. A research team conducted a four-year follow-up study on approximately 4,000 adults aged 40 to 69. As a result, people who snored six days or more per week had a 1.68 times higher probability of developing chronic bronchitis than those who did not. In particular, in the case of smokers, when snoring occurred six days or more per week, a risk of developing chronic bronchitis was 2.9 times higher than that of normal individuals.

### BRIEF SUMMARY OF THE INVENTION

The present invention provides a method and system for inducing sleep posture capable of inducing a sleep posture through a body-attached wearable sensor in order to prevent snoring and/or sleep apnea during sleep.

The present invention provides a system for inducing sleep posture including a sensor device attached to a body of a user and configured to collect first data including at least one of posture data of the user and respiration data of the user and transmit the collected first data to a user device, and configured to generate vibration for inducing a change in a sleep posture of the user in response to a control signal received from the user device; and the user device configured to collect second data including sound information related to sleep of the user, aggregate and analyze the first data received from the sensor device and the second data to determine a snoring state or a sleep apnea state of the user, and transmit, to the sensor device, the control signal for generating vibration in response to a determination that the user is in the snoring state or the sleep apnea state.

According to one aspect, the user device may determine a snoring-related value or a sleep apnea-related value through analysis of the first data and the second data, and may determine that the user is in the snoring state or the sleep apnea state when the value is equal to or greater than a preset threshold.

According to another aspect, the respiration data may be generated by continuously measuring, based on at least one of a change in a resonant frequency generated through an oscillator and repetitive charging and discharging of a sensor included in the sensor device and attached to the body of the user, a change in a fringing field formed through the sensor according to respiration activity of the user.

According to another aspect, the second data may include at least one of a respiration sound of the user, a snoring sound of the user, and a sound generated according to movement of the user during sleep, which are collected through a microphone included in the user device.

The present invention provides a sensor device attached to a body of a user, including a sensing unit configured to collect first data including at least one of posture data of the user and respiration data of the user and second data including sound information related to sleep of the user; a control unit configured to aggregate and analyze the first data and the second data to determine a snoring state or a sleep apnea state of the user, and generate a control signal for generating vibration in response to a determination that the user is in the snoring state or the sleep apnea state; and an output unit configured to generate vibration according to the control signal.

According to one aspect, the control unit may determine a snoring-related value or a sleep apnea-related value through analysis of the first data and the second data, and may determine that the user is in the snoring state or the sleep apnea state when the value is equal to or greater than a preset threshold.

According to another aspect, the sensing unit may include a sensor attached to a body of the user and a measurement circuit configured to measure sensing data of the sensor, and the measurement circuit may generate the respiration data by continuously measuring, based on at least one of a change in a resonant frequency generated through an oscillator and repetitive charging and discharging of the sensor, a change in a fringing field formed through the sensor according to respiration activity of the user.

According to still another aspect, the sensing unit may include a microphone, and the second data may include at least one of a respiration sound of the user, a snoring sound of the user, and a sound generated according to movement of the user during sleep, which are collected through the microphone.

The present invention provides a method for inducing sleep posture performed by a sensor device attached to a body of a user, the method including collecting first data including at least one of posture data of the user and respiration data of the user; transmitting the first data to a user device through a communication unit included in the sensor device; receiving a control signal from the user device through the communication unit; and generating vibration according to the control signal through an output unit included in the sensor device, wherein the user device collects second data including sound information related to sleep of the user, aggregates and analyzes the first data and the second data to determine a snoring state or a sleep apnea state of the user, and generates and transmits, to the sensor device, the control signal for generating vibration in response to a determination that the user is in the snoring state or the sleep apnea state.

The present invention provides a method for inducing sleep posture performed by a sensor device attached to a body of a user, the method including collecting first data including at least one of posture data of the user and respiration data of the user; collecting second data including sound information related to sleep of the user through a microphone included in the sensor device; under control of a control unit included in the sensor device, aggregating and analyzing the first data and the second data to determine a snoring state or a sleep apnea state of the user; under control of the control unit, generating a control signal for generating vibration in response to a determination that the user is in the snoring state or the sleep apnea state; and generating vibration according to the control signal through an output unit included in the sensor device.

Sleep posture may be induced through a body-attached wearable sensor in order to prevent snoring and/or sleep apnea during sleep.

### BRIEF DESCRIPTION OF THE DRAWINGS

FIG. 1 is a diagram illustrating an example of an overall configuration of a system for inducing sleep posture according to one embodiment of the present invention.

FIG. 2 is a diagram illustrating an example of an internal configuration of a sensor device according to one embodiment of the present invention.

FIG. 3 is a diagram illustrating an example of changes in a sensor device according to respiration activity of a user in one embodiment of the present invention.

FIG. 4 is a diagram illustrating an example of a fringing field in one embodiment of the present invention.

FIG. 5 is a diagram illustrating an example of a measurement circuit according to one embodiment of the present invention.

FIG. 6 is a diagram illustrating an example of an operation of a clock counter in one embodiment of the present invention.

FIG. 7 is a diagram illustrating another example of a measurement circuit according to one embodiment of the present invention.

FIG. 8 is a diagram illustrating an example of an operation of an ADC in one embodiment of the present invention.

FIG. 9 is a flowchart illustrating an example of a method for inducing sleep posture according to one embodiment of the present invention.

FIG. 10 is a flowchart illustrating another example of a method for inducing sleep posture according to one embodiment of the present invention.

FIG. 11 is a block diagram illustrating an example of a computer device according to one embodiment of the present invention.

### DETAILED DESCRIPTION OF THE INVENTION

Hereinafter, embodiments will be described in detail with reference to the accompanying drawings. However, various changes may be made to the embodiments, and thus the claims of the patent application are not limited or limited by these embodiments. It should be understood that all changes, equivalents, and alternatives to the embodiments are included in the claims.

Terms used in the embodiments are used for the purpose of description only, and should not be construed as limiting. The singular forms “a”, “an”, and “the” include plural forms unless the context clearly dictates otherwise. It should be understood that the terms “comprises”, “comprising”, “includes”, “including”, “having” and the like in this specification specify the presence of stated features, numbers, steps, operations, components, parts or combinations thereof, but do not preclude the presence or addition of one or more other features, numbers, step, operations, components or combinations thereof.

Unless otherwise defined, all terms used herein, including technical or scientific terms, have the same meaning as commonly understood by one of ordinary skill in the art to which the embodiments belong. Terms, such as those defined in commonly used dictionaries, should be interpreted as having a meaning that is consistent with their meaning in the context of the relevant art and will not be interpreted in an idealized or overly formal sense unless expressly so defined herein.

Furthermore, in the description with reference to the accompanying drawings, the same components are given the same reference numerals regardless of the drawing reference numerals, and redundant descriptions thereof will be omitted. In describing the embodiments, when it is determined that detailed descriptions of related known technologies may unnecessarily obscure the gist of the embodiments, such detailed descriptions will be omitted.

Furthermore, in describing components of the embodiments, terms such as first, second, A, B, (a), and (b) may be used. Such terms are used only for the purpose of distinguishing one component from another and do not limit the nature, order, or sequence of the corresponding components. When a component is described as being “connected,” “coupled,” or “linked” to another component, it should be understood that the component may be directly connected, coupled, or linked to the other component, or one or more other components may be connected, coupled, or linked between the respective components.

Components included in any one embodiment and components having common functions will be described using the same names in other embodiments. Unless otherwise stated, descriptions provided in any one embodiment may be applied to other embodiments, and detailed descriptions will be omitted in overlapping ranges.

As a lifestyle habit for preventing snoring and sleep apnea, “sleeping on one's side” is known. When sleeping in a supine position, a throat may become further narrowed because a tongue is pushed backward due to gravity. Accordingly, it is recommended to use a low pillow to prevent a neck from bending or to sleep while lying on one's side. In fact, according to a study, it has been shown that snoring and sleep apnea can be reduced by up to 80% simply by changing a sleep posture.

Embodiments of the present invention provide a method and system for inducing sleep posture for inducing a change in a sleep posture of a user during sleep.

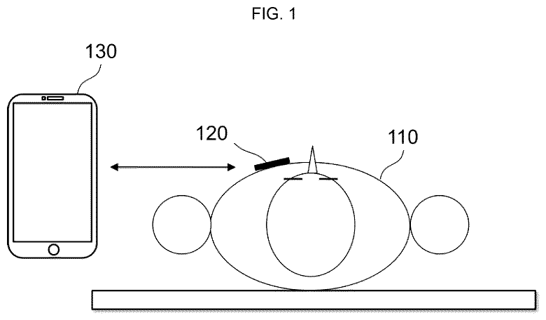

FIG. 1 is a diagram illustrating an example of an overall configuration of a system for inducing sleep posture according to one embodiment of the present invention. FIG. 1 illustrates examples of a user 110, a sensor device 120 attached to a body of the user 110, and a user device 130 of the user 110. The system for inducing sleep posture according to one embodiment may include the sensor device 120 and the user device 130.

The sensor device 120 may be attached to a specific body part of the user 110, such as a thorax, but is not limited thereto. The sensor device 120 may collect first data related to posture and/or respiration of the user 110 and may wirelessly communicate with the user device 130. For example, the sensor device 120 may transmit the collected first data of the user 110 to the user device 130. The first data collected by the sensor device 120 may include posture data and/or respiration data. As an example, the sensor device 120 may include an accelerometer and/or a gyroscope, and may collect outputs of the accelerometer and/or the gyroscope included in the sensor device 120 as the posture data. As another example, the sensor device 120 may collect respiration data such as a respiration pattern and/or a respiration cycle of the user 110 by continuously measuring changes according to respiration activity of the user 110. Collection of such respiration data will be described in further detail below.

The user device 130 may collect second data. As an example, the user device 130 may collect the second data through sound signals input through a microphone. As an example, the second data may include a respiration sound of the user 110, a snoring sound, and/or a sound generated according to movement of the user 110 during sleep.

In this case, the user device 130 may aggregate and analyze the first data collected and transmitted by the sensor device 120 and the second data collected by the user device 130 to detect snoring and/or sleep apnea information of the user 110. As an example, the user device 130 may determine a snoring-related value and/or a sleep apnea-related value as a result of aggregating and analyzing the first data and the second data. In this case, when the determined value is equal to or greater than a preset threshold, the user device 130 may determine that the user 110 is in a snoring state and/or a sleep apnea state.

In this case, the user device 130 may transmit a control signal to the sensor device 120. The sensor device 120 may generate vibration according to the control signal transmitted from the user device 130 to induce a change in a sleep posture of the user 110. Intensity and/or a pattern of vibration of the sensor device 120 may be adjusted according to settings of the user 110 and/or according to the value determined for snoring and/or sleep apnea.

A computer program in the form of an application for a sleep posture induction service may be installed and executed in the user device 130, and the user device 130 may operate under control of the computer program.

In addition, according to an embodiment, the sensor device 120 may include functions of the user device 130. In this case, the system for inducing sleep posture may include the sensor device 120. For example, the sensor device 120 may further collect the second data by including a microphone to collect a respiration sound of the user 110, a snoring sound, and sounds generated according to movement of the user 110 during sleep.

In this case, the sensor device 120 may aggregate and analyze the first data and the second data collected by the sensor device 120 to determine a snoring-related value and/or a sleep apnea-related value. In this case, when the determined value is equal to or greater than a preset threshold, the sensor device 120 may determine that the user 110 is in a snoring state and/or a sleep apnea state. Thereafter, the sensor device 120 may generate vibration in response to a determination that the user 110 is in the snoring state and/or the sleep apnea state to induce a change in a sleep posture of the user 110. Intensity and/or a pattern of vibration of the sensor device 120 may be adjusted according to settings of the user 110 and/or according to the value determined for snoring and/or sleep apnea.

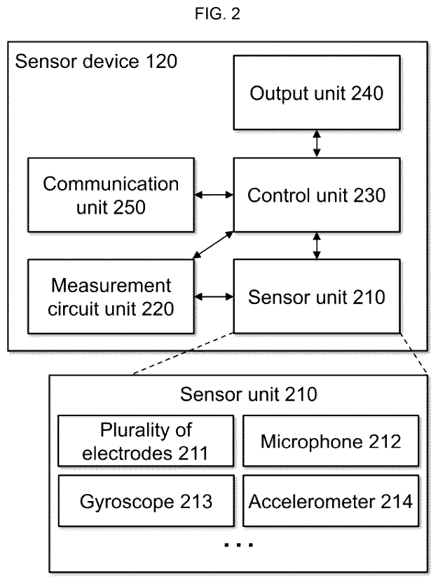

FIG. 2 is a diagram illustrating an example of an internal configuration of a sensor device according to one embodiment of the present invention. The sensor device 120 according to the embodiment of FIG. 2 may include a sensing unit 210, a measurement circuit 220, a control unit 230, an output unit 240, and a communication unit 250.

The sensing unit 210 may include various sensors such as a plurality of electrodes 211, a microphone 212, a gyroscope 213, and/or an accelerometer 214, as shown in FIG. 2.

Here, the plurality of electrodes 211 may be electrodes for forming a fringing field to be described later, and may be used to obtain respiration data of the user 110 that may be included in the first data. The plurality of electrodes 211 will be described in further detail below.

In addition, the microphone 212 may be included to collect the second data such as a respiration sound of the user 110, a snoring sound, and sounds generated according to movement of the user 110 during sleep in an embodiment in which the user device 130 is not used.

The gyroscope 213 and/or the accelerometer 214 may be used to obtain posture data of the user 110 that may be included in the first data.

Components of the sensing unit 210 may be partially omitted or additional components may be added according to an embodiment. For example, in order to obtain ambient environment information related to snoring and/or sleep apnea of the user 110, the sensing unit 210 may further include a temperature sensor, a humidity sensor, or the like.

The measurement circuit 220 may include a measurement circuit configured to read sensor data (or sensing data) through the sensing unit 210. As an example, the measurement circuit 220 may read respiration data based on the plurality of electrodes 211. The measurement circuit 220 will be described in further detail below.

The control unit 230 may control operations of the sensing unit 210, the measurement circuit 220, and the output unit 240, and may control the communication unit 250 to transmit measured data to the user device 130 or to receive setting values (for example, setting values for intensity and/or pattern of vibration) from the user device 130. The communication unit 250 may include a communication module for wired or wireless connection (preferably wireless connection) with the user device 130. Data communication between the communication unit 250 and the user device 130 may be performed using at least one of various well-known communication protocols such as BLE (Bluetooth Low Energy), NFC (Near Field Communication), and WiFi.

The output unit 240 may output vibration having a specific pattern under control of the control unit 230. As an example, the output unit 240 may include a motor, and the control unit 230 may control the motor to control the output unit 240 such that vibration having a specific intensity and/or pattern is output. Here, generating vibration with a specific intensity and/or pattern may mean that at least one of whether to generate the vibration and a magnitude of the vibration is adjusted for a predetermined period of time.

With respect to the respiration data, the user device 130 may be a user terminal such as a smartphone or a smartwatch. As an example, with respect to the respiration data, the user device 130 may display data collected by the sensor device 120 (for example, waveform data), and may display a respiration rate, a respiration quality, and a sleep quality of the user 110 determined based on the collected data. For this purpose, an algorithm for determining the respiration rate, the respiration quality, and the sleep quality may be executed in the sensor device 120 or the user device 130. When the algorithm is executed in the sensor device 120, the sensor device 120 may further transmit, to the user device 130, information on the respiration rate, the respiration quality, and the sleep quality of the user 110 determined using the collected data in addition to the collected data.

As one embodiment, in a case in which the system for inducing sleep posture includes both the sensor device 120 and the user device 130, the sensor device 120 may be attached to a body of the user 110 and may collect first data (posture data and/or respiration data) of the user 110 using the sensing unit 210 and/or the measurement circuit 220 under control of the control unit 230. In this case, the sensor device 120 may transmit the collected first data to the user device 130 through the communication unit 250 under control of the control unit 230.

Meanwhile, the user device 130 may collect second data including sound information related to sleep of the user 110. In this case, the user device 130 may aggregate and analyze the first data received from the sensor device 120 and the second data collected by the user device 130 to detect snoring and/or sleep apnea information of the user 110. As an example, the user device 130 may determine a snoring-related value and/or a sleep apnea-related value as a result of aggregating and analyzing the first data and the second data. In this case, when the determined value is equal to or greater than a preset threshold, the user device 130 may determine that the user 110 is in a snoring state and/or a sleep apnea state. In this case, the user device 130 may transmit a control signal to the sensor device 120.

The sensor device 120 may generate vibration according to the control signal transmitted from the user device 130 to induce a change in a sleep posture of the user 110. Intensity and/or a pattern of vibration of the sensor device 120 may be adjusted according to settings of the user 110 and/or according to the value determined for snoring and/or sleep apnea.

In this case, the sensor device 120 may generate vibration through the output unit 240 according to the control signal received from the user device 130 under control of the control unit 230, thereby inducing a change in the sleep posture of the user 110.

As another embodiment, in a case in which the system for inducing sleep posture does not include the user device 130 and includes the sensor device 120 to induce a change in a sleep posture of the user 110, the sensor device 120 may collect first data (posture data and/or respiration data) and/or second data (such as respiration sounds, snoring sounds, and/or sounds generated according to movement of the user 110 during sleep) using the sensing unit 210 and/or the measurement circuit 220. In this case, the sensor device 120 may aggregate and analyze the collected first data and/or second data through the control unit 230 to detect snoring and/or sleep apnea information of the user 110. As an example, the sensor device 120 may determine a snoring-related value and/or a sleep apnea-related value as a result of aggregating and analyzing the first data and the second data. In this case, when the determined value is equal to or greater than a preset threshold, the sensor device 120 may determine that the user 110 is in a snoring state and/or a sleep apnea state. In this case, the sensor device 120 may generate vibration to induce a change in a sleep posture of the user 110. As described above, intensity and/or a pattern of vibration of the sensor device 120 may be adjusted according to settings of the user 110 and/or according to the value determined for snoring and/or sleep apnea.

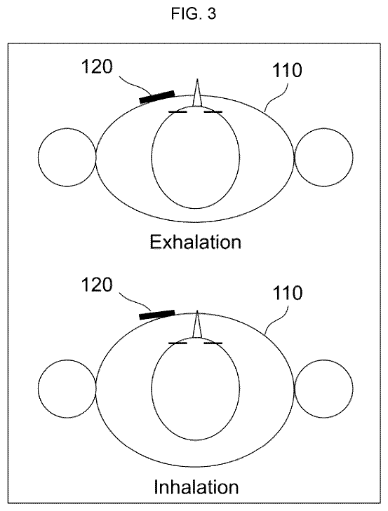

FIG. 3 is a diagram illustrating an example of changes in a sensor device according to respiration activity of a user in one embodiment of the present invention. FIG. 3 illustrates an example of a sensor device 120 attached to a user 110 performing respiration activity. A body part such as a thorax of the user 110 undergoes movement as its volume changes according to respiration activity of the user 110.

The sensor device 120 may be attached to such a specific body part of the user 110. In this case, the sensor device 120 may be attached so as not to be completely in close contact with an outer surface of the user 110. For example, in the case of a human body, the sensor device 120 may be attached such that only a portion of one surface of the sensor device 120 is attached to skin of the human body, thereby preventing the entire surface of the sensor device 120 from being in close contact with the skin of the human body.

In this case, when the user 110 performs respiration activity, movement occurs as a volume of a thorax changes, and according to such movement, a degree of contact between the sensor device 120 and the outer surface of the user 110 continuously changes, thereby inducing a certain variation. In the embodiment of FIG. 3, it is illustrated that the degree of contact between the sensor device 120 and the user 110 differs during inhalation and exhalation of the user 110.

In this case, the sensor device 120 may measure respiration data such as a respiration pattern and/or a respiration cycle of the user 110 by continuously measuring changes according to respiration activity of the user 110.

In one embodiment, the sensor device 120 may form a fringing field extending into an interior of a surface of the user 110 by using two or more electrodes. According to an embodiment, the fringing field may be formed to reach at least the surface of the user 110. In this case, the sensor device 120 may obtain information on respiration of the user 110 by measuring changes in the fringing field according to respiration activity of the user 110. In this case, as a method for measuring changes in the fringing field according to respiration activity of the user 110, an oscillator and/or repetitive charging and discharging of the sensor device 120 may be utilized.

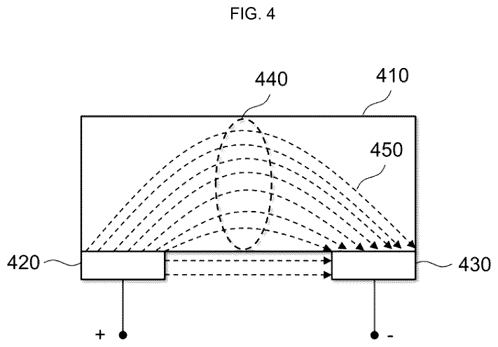

FIG. 4 is a diagram illustrating an example of a fringing field in one embodiment of the present invention. FIG. 4 illustrates two electrodes 420, 430 attached to a MUT (Material Under Test) 410. In this case, as a voltage is applied to the two electrodes 420, 430, a fringing field 440 may be formed into the MUT 410 between the two electrodes 420, 430, as illustrated in FIG. 4. The two electrodes 420, 430 may be an example of the plurality of electrodes 211 described above. In FIG. 4, the fringing field 440 is illustrated as a dotted ellipse for ease of understanding; however, in practice, the fringing field 440 may be formed by electromagnetic field lines (for example, field lines 450 of FIG. 4) between two conductors when a voltage is biased to a capacitor.

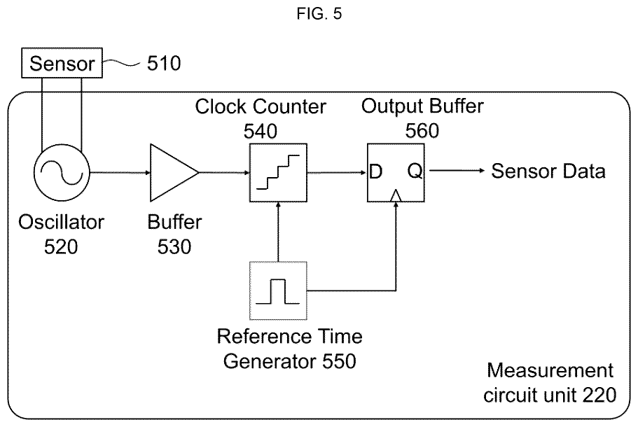

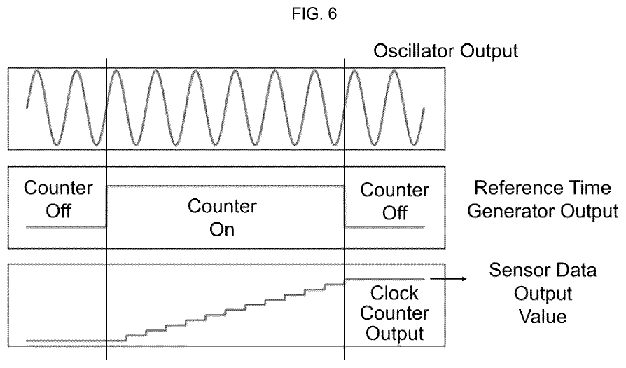

FIG. 5 is a diagram illustrating an example of a measurement circuit according to one embodiment of the present invention, and FIG. 6 is a diagram illustrating an example of an operation of a clock counter in one embodiment of the present invention. The measurement circuit 220 according to the embodiment of FIG. 5 may include a circuit for measuring respiration data.

The measurement circuit 220 may include an oscillator 520 connected to a sensor 510, a buffer 530, a clock counter 540, a reference time generator 550, and an output buffer 560.

The sensor 510 may correspond to the plurality of electrodes 211 described above. For example, as a voltage is applied to at least two electrodes included in the sensor 510 (for example, the two electrodes 420, 430), a fringing field may be formed. The oscillator 520 may be an RC (Resistor-Capacitor) oscillator or an LC (Inductor-Capacitor) oscillator. In this case, when the fringing field changes according to respiration, an output frequency (resonant frequency) of the oscillator 520 connected to the sensor 510 may change. In this case, an output signal of the oscillator 520 may be input to the clock counter 540 through the buffer 530.

The clock counter 540 may count cycles of an input signal during a reference time generated by the reference time generator 550. As a frequency of the input signal increases, a relatively greater number of cycles may be counted during the reference time, and thus an output value of the clock counter 540 may increase. The reference time generator 550 may generate a signal defining the reference time during which the clock counter 540 operates.

An output of the clock counter 540 may be output as sensor data through the output buffer 560.

FIG. 6 illustrates an example in which, when an output of the oscillator 520 (a resonant frequency signal) is input to the clock counter 540, the clock counter 540 counts cycles of the output of the oscillator 520 according to an output of the reference time generator 550 and outputs the counted result as an output value of sensor data.

As described above, according to respiration of the user 110, a fringing field formed through the sensor 510 changes, an oscillation resonant frequency output by the oscillator 520 changes according to the change in the fringing field, and an output value of the clock counter 540 may change according to the change in the resonant frequency. Accordingly, information on respiration of the user 110 (respiration data) may be obtained based on a change in the output value of the clock counter 540.

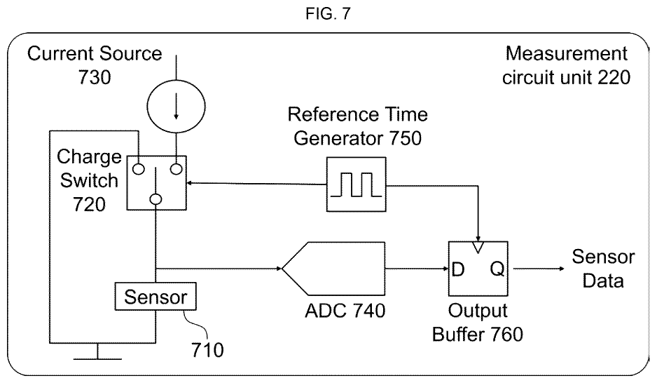

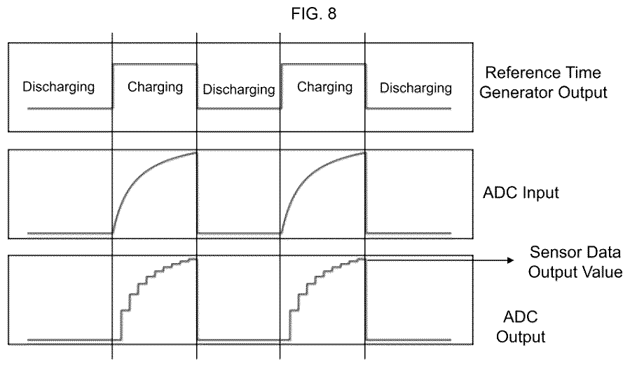

FIG. 7 is a diagram illustrating another example of a measurement circuit according to one embodiment of the present invention, and FIG. 8 is a diagram illustrating an example of an operation of an ADC in one embodiment of the present invention.

The measurement circuit 220 according to the embodiment of FIG. 7 may include a charge switch 720 connected to a sensor 710, a current source 730, an ADC 740, a reference time generator 750, and an output buffer 760.

The sensor 710 may correspond to the plurality of electrodes 211 described above. In the embodiment of FIG. 7, the sensor 710 is illustrated as being included in the measurement circuit 220; however, in practice, the sensor 710 may be disposed outside the measurement circuit 220 so as to be attached to the user 110.

The measurement circuit 220 may measure a degree of charging while repeatedly charging and discharging the sensor 710 using the charge switch 720. The reference time generator 750 may generate a control signal having a reference time interval to operate the charge switch 720. When the charge switch 720 is turned on, the sensor 710 and the current source 730 are connected such that the sensor 710 may be charged, and when the charge switch 720 is turned off, the connection between the sensor 710 and the current source 730 is released such that the sensor 710 may be discharged.

While the sensor 710 is being charged, an input terminal voltage of the sensor 710 may increase, and the measurement circuit 220 may convert the voltage into a digital code using the ADC 740. In this case, an output value of the ADC 740 at a time point at which the charge switch 720 is turned off by the reference time generator 750 may be output as sensor data through the output buffer 760.

In FIG. 8, inputs and outputs of the ADC 740 are respectively illustrated according to repetition of connection and disconnection between the sensor 710 and the current source 730 by the charge switch 720 in response to an output of the reference time generator 750. In addition, FIG. 8 illustrates that an output value of the ADC 740 at a time point at which the charge switch 720 is turned off by the reference time generator 750 may be output as a sensor data output value.

As described above, as a fringing field changes according to respiration of the user 110, a degree to which the sensor 710 is charged changes, and an output value of the ADC 740 may change according to the change in the degree of charging of the sensor 710. Accordingly, information on respiration of the user 110 (respiration data) may be obtained based on a change in the output value of the ADC 740.

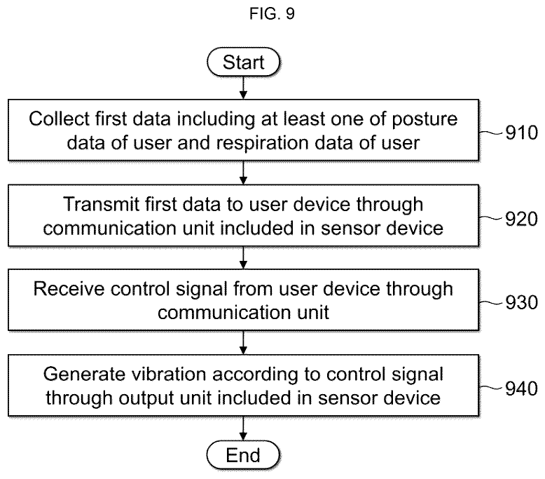

FIG. 9 is a flowchart illustrating an example of a method for inducing sleep posture according to one embodiment of the present invention. The method for inducing sleep posture according to the present embodiment may be performed by the sensor device 120 described above. As one embodiment, the control unit 230 of the sensor device 120 may include at least one processor and a memory. In this case, operation of the sensor device 120 may be interpreted as being implemented as the processor of the control unit 230 controls the sensing unit 210, the measurement circuit 220, and the communication unit 250 included in the sensor device 120 according to code of a computer program stored in the memory of the control unit 230.

In step 910, the sensor device 120 may collect first data including at least one of posture data and respiration data of the user 110. As one embodiment, the sensor device 120 may include a sensing unit configured to collect the first data including at least one of the posture data and the respiration data of the user 110. The sensing unit herein may include the sensing unit 210 and the measurement circuit 220 described above with reference to FIG. 2. In this case, the sensing unit may include, as a part of the sensing unit 210, a sensor attached to a body of the user 110 (for example, the sensor 510 and/or the sensor 710), and the measurement circuit 220 may measure sensing data of the sensor. In this case, the measurement circuit 220 may generate the respiration data by continuously measuring, based on at least one of a change in a resonant frequency generated through an oscillator and repetitive charging and discharging of the sensor, a change in a fringing field formed through the sensor according to respiration activity of the user 110. The posture data of the user 110 may include outputs of an accelerometer and/or a gyroscope, as described above.

In step 920, the sensor device 120 may transmit the first data to the user device 130 through the communication unit 250 included in the sensor device 120. In this case, the user device 130 may collect second data including sound information related to sleep of the user 110, and may determine a snoring state or a sleep apnea state of the user 110 by aggregating and analyzing the first data and the second data. In this case, the user device 130 may determine a value for snoring or a value for sleep apnea through analysis of the first data and the second data, and may determine the user to be in the snoring state or the sleep apnea state when the value is equal to or greater than a preset threshold value. In this case, the user device 130 may generate a control signal for generation of vibration in response to a determination that the user 110 is in the snoring state or the sleep apnea state, and may transmit the control signal to the sensor device 120. Here, the second data may include at least one of a respiration sound of the user 110, a snoring sound of the user 110, and a sound generated according to movement of the user 110 during sleep, which are collected through a microphone included in the user device 130.

In step 930, the sensor device 120 may receive the control signal from the user device 130 through the communication unit 250.

In step 940, the sensor device 120 may generate vibration according to the control signal through the output unit 240 included in the sensor device 120. An intensity and/or a pattern of the vibration may be adjusted according to a setting of the user 110 and/or according to a value determined for snoring and/or sleep apnea. The generated vibration may induce a change in a sleep posture of the user, and snoring and/or sleep apnea may be prevented through the change in the sleep posture.

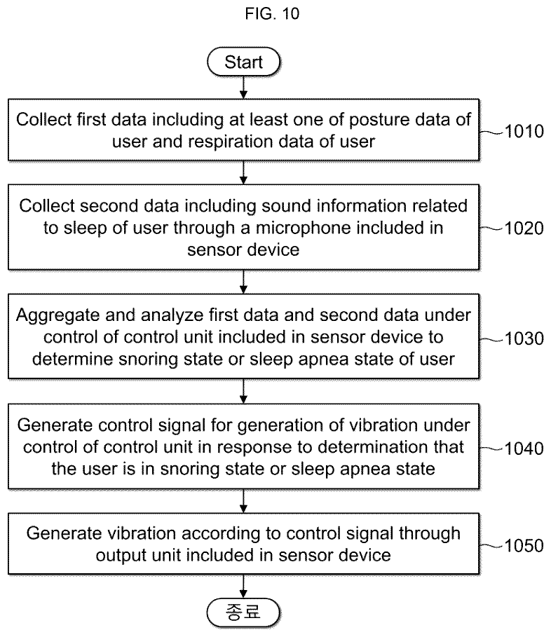

FIG. 10 is a flowchart illustrating another example of a method for inducing sleep posture according to one embodiment of the present invention. The method for inducing sleep posture according to the present embodiment may be performed by the sensor device 120 described above. As one embodiment, the control unit 230 of the sensor device 120 may include at least one processor and a memory. In this case, operation of the sensor device 120 may be interpreted as being implemented as the processor of the control unit 230 controls the sensing unit 210, the measurement circuit 220, and the communication unit 250 included in the sensor device 120 according to code of a computer program stored in the memory of the control unit 230.

In step 1010, the sensor device 120 may collect first data including at least one of posture data of the user and respiration data of the user. As described above, as one embodiment, the sensor device 120 may include a sensing unit configured to collect the first data including at least one of the posture data and the respiration data of the user 110. The sensing unit herein may include the sensing unit 210 and the measurement circuit 220 described above with reference to FIG. 2. In this case, the sensing unit may include, as a part of the sensing unit 210, a sensor attached to a body of the user 110 (for example, the sensor 510 and/or the sensor 710), and the measurement circuit 220 may measure sensing data of the sensor. In this case, the measurement circuit 220 may generate the respiration data by continuously measuring, based on at least one of a change in a resonant frequency generated through an oscillator and repetitive charging and discharging of the sensor, a change in a fringing field formed through the sensor according to respiration activity of the user 110. The posture data of the user 110 may include outputs of an accelerometer and/or a gyroscope, as described above.

In step 1020, the sensor device 120 may collect second data including sound information related to sleep of the user 110 through the microphone 212 included in the sensor device 120. Here, the second data may include at least one of a respiration sound of the user 110, a snoring sound of the user 110, and a sound generated according to movement of the user 110 during sleep, which are collected through the microphone 212.

In step 1030, the sensor device 120 may, under control of the control unit 230 included in the sensor device 120, aggregate and analyze the first data and the second data to determine a snoring state or a sleep apnea state of the user 110. As an example, the control unit 230 may determine a value for snoring or a value for sleep apnea through analysis of the first data and the second data, and may determine the user 110 to be in the snoring state or the sleep apnea state when the value is equal to or greater than a preset threshold value.

In step 1040, the sensor device 120 may, under control of the control unit 230, generate a control signal for generation of vibration in response to a determination that the user 110 is in the snoring state or the sleep apnea state. According to an embodiment, the control signal may include information on an intensity of vibration and/or a pattern of vibration.

In step 1050, the sensor device 120 may generate vibration according to the control signal through the output unit 240 included in the sensor device 120. The intensity and/or the pattern of the vibration may be adjusted according to a setting of the user 110 and/or according to a value determined for snoring and/or sleep apnea. The generated vibration may induce a change in a sleep posture of the user, and snoring and/or sleep apnea may be prevented through the change in the sleep posture.

Meanwhile, the user device 130 may be implemented by, for example, a computer device.

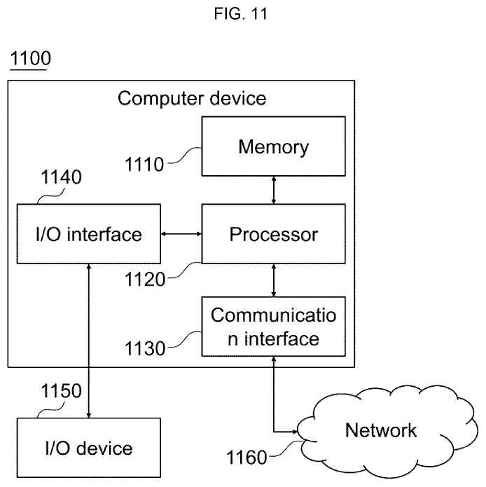

FIG. 11 is a block diagram illustrating an example of a computer device according to one embodiment of the present invention. As illustrated in FIG. 11, a computer device 1100 may include a memory 1110, a processor 1120, a communication interface 1130, and an I/O interface 1140. The memory 1110 may be a computer-readable recording medium and may include RAM (random access memory), ROM (read only memory), and non-volatile mass storage devices such as disk drives (permanent mass storage devices). Here, non-volatile mass storage devices such as ROM and disk drives may be included in the computer device 1100 as separate permanent storage devices distinguished from the memory 1110. In addition, the memory 1110 may store an operating system and at least one program code. Such software components may be loaded into the memory 1110 from a computer-readable recording medium separate from the memory 1110. Such separate computer-readable recording media may include computer-readable recording media such as floppy drives, disks, tapes, DVD/CD-ROM drives, and memory cards. In another embodiment, the software components may be loaded into the memory 1110 through the communication interface 1130 rather than through a computer-readable recording medium. For example, the software components may be loaded into the memory 1110 of the computer device 1100 based on a computer program installed by files received through a network 1160.

The processor 1120 may be configured to process instructions of a computer program by performing basic arithmetic, logic, and input/output operations. The instructions may be provided to the processor 1120 by the memory 1110 or the communication interface 1130. For example, the processor 1120 may be configured to execute instructions received according to program code stored in a recording device such as the memory 1110.

The communication interface 1130 may provide a function for enabling the computer device 1100 to communicate with other devices through the network 1160. As an example, requests, commands, data, files, and the like generated by the processor 1120 of the computer device 1100 according to program code stored in a recording device such as the memory 1110 may be transmitted to other devices through the network 1160 under control of the communication interface 1130. Conversely, signals, commands, data, files, and the like from other devices may be received by the computer device 1100 through the communication interface 1130 of the computer device 1100 via the network 1160. Signals, commands, data, and the like received through the communication interface 1130 may be delivered to the processor 1120 or the memory 1110, and files and the like may be stored in a storage medium (the above-described permanent storage device) further included in the computer device 1100.

The input/output interface 1140 may be a means for interfacing with an I/O device 1150. For example, an input device may include a microphone, a keyboard, or a mouse, and an output device may include a display or a speaker. As another example, the input/output interface 1140 may be a means for interfacing with a device in which functions for input and output are integrated into a single device, such as a touchscreen. The input/output device 1150 may be configured as a single device together with the computer device 1100.

In addition, in other embodiments, the computer device 1100 may include fewer or more components than those illustrated in FIG. 11. However, it is not necessary to clearly illustrate most conventional components. For example, the computer device 1100 may be implemented to include at least a part of the above-described input/output device 1150, or may further include other components such as a transceiver or a database.

As described above, according to embodiments of the present invention, a sleep posture may be induced through a body-attached wearable sensor in order to prevent snoring and/or sleep apnea during sleep.

The system or device described above may be implemented using hardware components, or a combination of hardware components and software components. For example, the devices and components described in the embodiments may be implemented using one or more general-purpose computers or special-purpose computers, such as a processor, a controller, an arithmetic logic unit (ALU), a digital signal processor (DSP), a microcomputer, a field programmable gate array (FPGA), a programmable logic unit (PLU), a microprocessor, or any other device capable of executing and responding to instructions. A processing device may execute an operating system (OS) and one or more software applications executed on the operating system. In addition, the processing device may access, store, manipulate, process, and generate data in response to execution of software. For convenience of understanding, a processing device may be described as being a single processing device; however, those skilled in the art will appreciate that the processing device may include a plurality of processing elements and/or a plurality of types of processing elements. For example, the processing device may include a plurality of processors, or a single processor and a single controller. In addition, other processing configurations, such as a parallel processor, are also possible.

Software may include a computer program, code, instructions, or a combination of one or more thereof, and may configure a processing device to operate as desired or may instruct the processing device independently or collectively. Software and/or data may be embodied in any type of machine, component, physical device, virtual equipment, computer storage medium, or apparatus in order to be interpreted by a processing device or to provide instructions or data to the processing device. The software may be distributed over network-connected computer systems and stored or executed in a distributed manner. The software and data may be stored in one or more computer-readable recording media.

A method according to an embodiment may be implemented in the form of program instructions executable through various computer means and recorded on a computer-readable medium. The computer-readable medium may include program instructions, data files, and data structures, either alone or in combination. The medium may continuously store a computer-executable program or temporarily store the program for execution or download. In addition, the medium may be various recording means or storage means in which one or several pieces of hardware are combined, and is not limited to a medium directly connected to a computer system, but may be distributed over a network. Examples of the medium may include magnetic media such as hard disks, floppy disks, and magnetic tapes; optical recording media such as CD-ROMs and DVDs; magneto-optical media such as floptical disks; and media configured to store program instructions, including ROM and flash memory. In addition, examples of other media may include recording media or storage media managed by app stores or other sites or servers that supply or distribute various software applications. Examples of program instructions may include not only machine language code generated by a compiler, but also high-level language code executable by a computer using an interpreter or the like.

Although the embodiments have been described above with reference to limited embodiments and drawings, various modifications and variations may be made from the above description by those skilled in the art. For example, the described techniques may be performed in an order different from the described methods, and/or components of the described systems, structures, devices, and circuits may be combined or integrated in forms different from those described, or may be replaced or substituted with other components or equivalents, and still achieve appropriate results.

Accordingly, other implementations, other embodiments, and equivalents to the claims are also intended to fall within the scope of the claims set forth below.

1. A system for inducing sleep posture, comprising:
a sensor device attached to a body of a user, the sensor device being configured to collect first data comprising at least one of posture data of the user and respiration data of the user and transmit the first data to a user device, and to generate vibration for inducing a change in a sleep posture of the user in response to a control signal received from the user device; and
the user device configured to collect second data comprising sound information related to sleep of the user, to aggregate and analyze the first data received from the sensor device and the second data to determine a snoring state or a sleep apnea state of the user, and to transmit, to the sensor device, the control signal for generation of vibration in response to a determination that the user is in the snoring state or the sleep apnea state,
wherein the respiration data is generated by continuously measuring, based on at least one of a change in a resonant frequency generated through an oscillator and repetitive charging and discharging of the sensor, a change in a fringing field formed through a sensor comprised in the sensor device and attached to the body of the user according to respiration activity of the user.

2. The system for inducing sleep posture of claim 1, wherein:
the user device determines a value for snoring or a value for sleep apnea through analysis of the first data and the second data, and determines the user to be in the snoring state or the sleep apnea state when the value is equal to or greater than a preset threshold value.

3. The system for inducing sleep posture of claim 1, wherein:
the second data comprises at least one of a respiration sound of the user, a snoring sound of the user, and a sound generated according to movement of the user during sleep, which are collected through a microphone comprised in the user device.

4. A sensor device attached to a body of a user, comprising:
a sensing unit configured to collect first data comprising at least one of posture data of the user and respiration data of the user and second data comprising sound information related to sleep of the user;
a control unit configured to aggregate and analyze the first data and the second data to determine a snoring state or a sleep apnea state of the user, and to generate a control signal for generation of vibration in response to a determination that the user is in the snoring state or the sleep apnea state; and
an output unit configured to generate vibration according to the control signal,
wherein the sensing unit comprises:
a sensor attached to the body of the user; and
a measurement circuit configured to measure sensing data of the sensor, and wherein the measurement circuit generates the respiration data by continuously measuring, based on at least one of a change in a resonant frequency generated through an oscillator and repetitive charging and discharging of the sensor, a change in a fringing field formed through the sensor according to respiration activity of the user.

5. The sensor device of claim 4, wherein:
the control unit determines a value for snoring or a value for sleep apnea through analysis of the first data and the second data, and determines the user to be in the snoring state or the sleep apnea state when the value is equal to or greater than a preset threshold value.

6. The sensor device of claim 4, wherein:
the sensing unit further comprises a microphone, and
the second data comprises at least one of a respiration sound of the user, a snoring sound of the user, and a sound generated according to movement of the user during sleep, which are collected through the microphone.

7. A method of operating a sensor device attached to a body of a user, comprising:
collecting first data comprising at least one of posture data of the user and respiration data of the user;
transmitting the first data to a user device through a communication unit comprised in the sensor device;
receiving a control signal from the user device through the communication unit; and
generating vibration according to the control signal through an output unit comprised in the sensor device,
wherein the user device collects second data comprising sound information related to sleep of the user, aggregates and analyzes the first data and the second data to determine a snoring state or a sleep apnea state of the user, and generates, in response to a determination that the user is in the snoring state or the sleep apnea state, the control signal for generation of vibration and transmits the control signal to the sensor device,
and the respiration data is generated by continuously measuring, based on at least one of a change in a resonant frequency generated through an oscillator and repetitive charging and discharging of a sensor comprised in the sensor device and attached to the body of the user, a change in a fringing field formed through the sensor according to respiration activity of the user.
